(19) United States
(12) Patent Application Publication
(10) Pub. No.: US 2021/0082577 A1
(43) Pub. Date: Mar. 18, 2021

(54) SYSTEM AND METHOD FOR PROVIDING USER-CUSTOMIZED PREDICTION MODELS AND HEALTH-RELATED PREDICTIONS BASED THEREON

(71) Applicant: KONINKLIJKE PHILIPS N.V., EINDHOVEN (NL)

(72) Inventors: Reza SEDEH SHARIFI, MALDEN, MA (US); AMIR MOHAMMAD TAHMASEBI MARAGHOOSH, ARLINGTON, MA (US); ANDREW G HOSS, CAMBRIDGE, MA (US)

(21) Appl. No.: 16/611,884

(22) PCT Filed: May 9, 2018
(86) PCT No.: PCT/EP2018/061949

§ 371 Date: Nov. 8, 2019

Related U.S. Application Data
(60) Provisional application No. 62/506,124, filed on May 15, 2017.

(51) Int. Cl. G16H 50/30; G06N 20/00; G06N 5/04

(52) U.S. Cl. CPC G16H 50/30; G06N 5/04; G06N 20/00

(57) ABSTRACT

The present system is configured to obtain training information related to patients, and obtain a user input indicating prediction criteria that are to be used by a prediction model for generating patient-related predictions. The prediction criteria include which and how many prediction-contributing features are to be used by the prediction model for generating patient-related predictions. The system is configured to generate the prediction model based on the prediction criteria and the training information; and generate, based on the prediction model and patient information associated with a patient, a prediction related to a health outcome of the patient. The system is also configured to cause display of the prediction and other predictions, wherein the display comprises a scaled display of two or more of the prediction-contributing features.

### TECHNICAL FIELD

The present disclosure pertains to a system and method for providing user-customized prediction models and health-related predictions based thereon.

### BACKGROUND

Present computing technology facilitates acquisition of, storage of, and access to, large amounts of data. As a result, a wide range of large healthcare databases are available for use by researchers and/or for other purposes. Researchers use various methods to extract and visualize data to gain insights regarding the impact of various data features on patients.

### SUMMARY

Accordingly, one or more aspects of the present disclosure relate to a system configured to provide user-customized prediction models and health-related predictions based thereon. The system comprises one or more hardware processors configured by machine readable instructions and/or other components. The system is configured to obtain training information related to patients. The training information comprises one or more of demographic information indicating demographics associated with the patients, vital signs information indicating vital signs associated with the patients, medical condition information indicating medical conditions experienced by the patients, treatment information indicating treatments received by the patients, or outcome information indicating health outcomes for the patients. The system is configured to obtain, via a user interface, a user input indicating prediction criteria that are to be used by a prediction model for generating patient-related predictions. The prediction criteria include which and how many prediction-contributing features that are to be used by the prediction model for generating patient-related predictions. The system is configured to generate the prediction model based on (i) the prediction criteria and (ii) the training information. The system is configured to generate, based on the prediction model and patient information associated with a patient, a prediction related to a health outcome of the patient.

In some embodiments, the system is configured to generate, based on the prediction model and patient information associated with other patients, predictions related to health outcomes of the other patients; and cause display of the prediction and the other predictions on the user interface, wherein the display of the prediction and the other predictions on the user interface comprises a scaled display of two or more of the prediction-contributing features used by the prediction model for generating the patient-related predictions relative to each other such that any change in a value of one of the two or more prediction-contributing features causes a corresponding scaled change in values of the others of the two or more prediction-contributing features.

Another aspect of the present disclosure relates to a method for providing user-customized prediction models and health-related predictions based thereon with a prediction system. The system comprises one or more hardware processors configured by machine readable instructions and/or other components. The method comprises: obtaining, with the one or more hardware processors, training information related to patients, the training information comprising one or more of demographic information indicating demographics associated with the patients, vital signs information indicating vital signs associated with the patients, medical condition information indicating medical conditions experienced by the patients, treatment information indicating treatments received by the patients, or outcome information indicating health outcomes for the patients; obtaining, with the one or more hardware processors via a user interface, a user input indicating prediction criteria that are to be used by a prediction model for generating patient-related predictions, the prediction criteria including which and how many prediction-contributing features are to be used by the prediction model for generating patient-related predictions; generating, with the one or more hardware processors, the prediction model based on (i) the prediction criteria and (ii) the training information; and generating, with the one or more hardware processors, based on the prediction model and patient information associated with a patient, a prediction related to a health outcome of the patient.

In some embodiments, the method comprises generating, with the one or more hardware processors, based on the prediction model and patient information associated with other patients, predictions related to health outcomes of the other patients; and causing, with the one or more hardware processors, display of the prediction and the other predictions on the user interface, wherein the display of the prediction and the other predictions on the user interface comprises a scaled display of two or more of the prediction-contributing features used by the prediction model for generating the patient-related predictions relative to each other such that any change in a value of one of the two or more prediction-contributing features causes a corresponding scaled change in values of the others of the two or more prediction-contributing features.

Still another aspect of present disclosure relates to a system for providing user-customized prediction models and health-related predictions based thereon. The system comprises: means for obtaining training information related to patients, the training information comprising one or more of demographic information indicating demographics associated with the patients, vital signs information indicating vital signs associated with the patients, medical condition information indicating medical conditions experienced by the patients, treatment information indicating treatments received by the patients, or outcome information indicating health outcomes for the patients; means for obtaining a user input indicating prediction criteria that are to be used by a prediction model for generating patient-related predictions, the prediction criteria including which and how many prediction-contributing features are to be used by the prediction model for generating patient-related predictions; means for generating the prediction model based on (i) the prediction criteria and (ii) the training information; and means for generating based on the prediction model and patient information associated with a patient, a prediction related to a health outcome of the patient.

In some embodiments, the system comprises means for generating, based on the prediction model and patient information associated with other patients, predictions related to health outcomes of the other patients; and means for causing display of the prediction and the other predictions on a user interface, wherein the display of the prediction and the other predictions on the user interface comprises a scaled display of two or more of the prediction-contributing features used by the prediction model for generating the patient-related predictions relative to each other such that any change in a value of one of the two or more prediction-contributing features causes a corresponding scaled change in values of the others of the two or more prediction-contributing features.

In some embodiments, a non-transitory machine-readable storage medium encoded with instructions for execution by a hardware processor, the non-transitory machine-readable storage medium including: instructions for obtaining training information related to patients, the training information including medical data of the patients; instructions for obtaining, via a user interface, a user input indicating prediction criteria that are to be used by a prediction model for generating patient-related predictions, the prediction criteria including how many prediction-contributing features are to be used by the prediction model for generating patient-related predictions and constraints on the prediction model; instructions for generating a prediction model including determining which prediction-contributing features to use in the prediction model based on the prediction criteria and the training information, wherein the prediction model predicts a health outcome of patients; and instructions for presenting the prediction model to the user.

Various embodiments are described, wherein the medical data of the patient includes one or more of demographic information indicating demographics associated with the patients, vital signs information indicating vital signs associated with the patients, medical condition information indicating medical conditions experienced by the patients, treatment information indicating treatments received by the patients, or outcome information indicating health outcomes for the patients.

Various embodiments are described, further including: instructions for receiving updated prediction criteria from the user after presenting the prediction mode to the user; and instructions for updating the prediction model based upon the updated prediction criteria.

Various embodiments are described, further including: instructions for receiving updated training data; and instructions for updating the prediction model based upon the updated training data.

Various embodiments are described, further including: instructions for displaying the predicted health outcome of a specific patient based upon the generated prediction model.

Various embodiments are described, wherein the prediction model is generated by machine learning by minimizing a 0-1 loss function for accuracy and a L0 norm regulation subject to the prediction criteria.

Various embodiments are described, further including: instructions for generating, based on the prediction model and patient information associated with other patients, predictions related to health outcomes of the other patients; and instructions for displaying prediction on a scaled display of two or more of the prediction-contributing features used by the prediction model for generating the patient-related predictions,

wherein the prediction model includes predictive-values feature scale factors that are used to scale display axes associated with the displayed prediction-contributing features.

Various embodiments are described, wherein presenting the prediction model to the user further comprises an initial prediction related to the health outcome of a patient presented via a numerical outcome risk score, a list of the prediction-contributing features, a mathematical relationship between the prediction-contributing features, and values of accuracy metrics corresponding to the two or more prediction-contributing features.

Various embodiments are described, wherein the constraints include one of target false positive outcome prediction rate associated with a given feature, a target degree of correlation between the given feature and the prediction related to the health outcome, indicating that one feature has a greater influence on the outcome than another feature, or a target amount the given feature influences the prediction related to the health outcome relative to other features.

Various further embodiments are described, including method, including obtaining training information related to patients, the training information including medical data of the patients; obtaining, via a user interface, a user input indicating prediction criteria that are to be used by a prediction model for generating patient-related predictions, the prediction criteria including how many prediction-contributing features are to be used by the prediction model for generating patient-related predictions and constraints on the prediction model; generating a prediction model including determining which prediction-contributing features to use in the prediction model based on the prediction criteria and the training information, wherein the prediction model predicts a health outcome of patients; and presenting the prediction model to the user.

Various embodiments are described, wherein, the medical data of the patient includes one or more of demographic information indicating demographics associated with the patients, vital signs information indicating vital signs associated with the patients, medical condition information indicating medical conditions experienced by the patients, treatment information indicating treatments received by the patients, or outcome information indicating health outcomes for the patients.

Various embodiments are described, further including: receiving updated prediction criteria from the user after presenting the prediction mode to the user; and updating the prediction model based upon the updated prediction criteria.

Various embodiments are described, further including: receiving updated training data; and updating the prediction model based upon the updated training data.

Various embodiments are described, further including: displaying the predicted health outcome of a specific patient based upon the generated prediction model.

Various embodiments are described, wherein the prediction model is generated by machine learning by minimizing a 0-1 loss function for accuracy and a L0 norm regulation subject to the prediction criteria.

Various embodiments are described, further including: generating, based on the prediction model and patient information associated with other patients, predictions related to health outcomes of the other patients; and displaying prediction on a scaled display of two or more of the prediction-contributing features used by the prediction model for generating the patient-related predictions, wherein the prediction model includes predictive-values feature scale factors that are used to scale display axes associated with the displayed prediction-contributing features.

Various embodiments are described, wherein presenting the prediction model to the user further comprises an initial prediction related to the health outcome of a patient presented via a numerical outcome risk score, a list of the prediction-contributing features, a mathematical relationship between the prediction-contributing features, and values of accuracy metrics corresponding to the two or more prediction-contributing features.

Various embodiments are described, wherein the constraints include one of target false positive outcome prediction rate associated with a given feature, a target degree of correlation between the given feature and the prediction related to the health outcome, indicating that one feature has a greater influence on the outcome than another feature, or a target amount the given feature influences the prediction related to the health outcome relative to other features.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 illustrates a system configured to provide user-customized prediction models and health-related predictions based thereon.

FIG. 2 illustrates a scatter plot of readmission risk values for a plurality of patients.

FIG. 3 illustrates operations performed by the system.

FIG. 4 illustrates a method for providing user-customized prediction models and health-related predictions based thereon with a prediction system.

### DETAILED DESCRIPTION

As used herein, the singular form of “a”, “an”, and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The extraction of meaningful information from these databases is a challenging task because visualization of data from high-dimension databases is difficult. For example, in a medical database that stores values for thousands of features for millions of patients, determining which features impact a specific health outcome (e.g., risk of readmission), much less visually presenting such information in a meaningful way, challenges typical computing systems. It may be difficult to choose a subset of the thousands of features on which to focus an analysis, or use to present visual information. It may be difficult to determine and/or understand the relative (compared to the thousands of other features) effect an individual feature has on a health outcome. Although some systems that analyze such large volumes of data may derive new features to represent data in fewer dimensions, the newly derived features of such systems may fail to be meaningful or fail to provide additional insight into the data. These and other drawbacks exist.

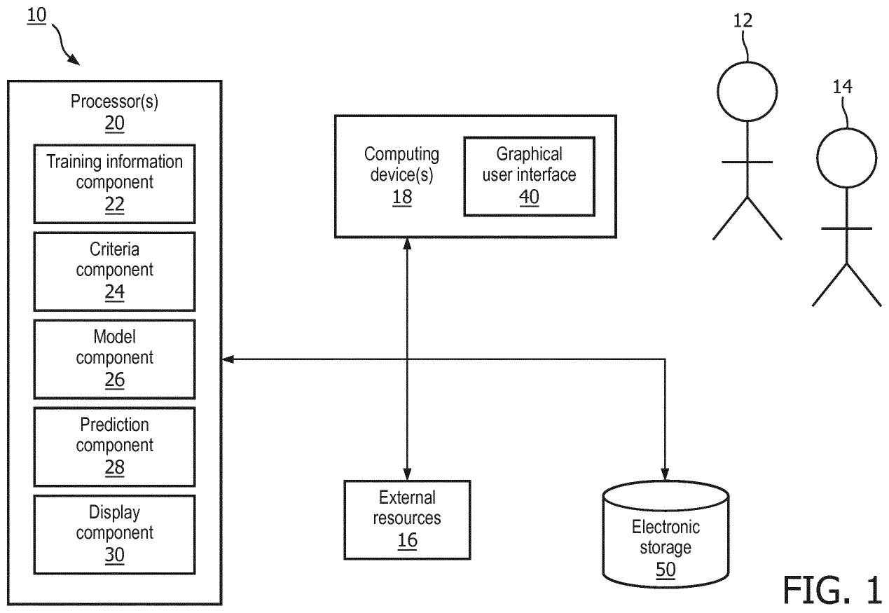

FIG. 1 is a schematic illustration of a system 10 configured to provide user-customized prediction models and health-related predictions based thereon. Advantageously, system 10 facilitates user entry, selection, and/or adjustment of a number of features used in a prediction model for a given health related prediction. In some embodiments the health related predictions may be health outcome predictions such as risk of readmission, mortality, length of stay, patient satisfaction, and/or other predictions. System 10 further facilitates user entry, selection, and/or adjustment of constraints and/or other criteria for the feature selection process for the prediction model. System 10 may facilitate insight into large datasets by correlating patient health outcomes and/or other information back to a select number of original features of the data (e.g., not new intermediate features formed by a combination of 10-20 or more of the original features as in prior art systems). System 10 may facilitate visualization of such high dimension data by using only the selected features and their influence on the health outcome to represent the data in a visualization. In some embodiments, system 10 includes one or more of external resources 16, computing devices 18, processors 20, electronic storage 50, and/or other components.

External resources 16 include sources of information and/or other resources. For example, external resources 16 may include training and/or other information. The training information may be related to patients 12. In some embodiments, the training information comprises demographic information indicating demographics associated with patients 12, vital signs information indicating vital signs associated with patients 12, medical condition information indicating medical conditions experienced by patients 12, treatment information indicating treatments received by patients 12, outcome information indicating health outcomes for patients 12, and/or other training information. In some embodiments, external resources 16 include sources of training information such as databases, websites, etc.; external entities participating with system 10 (e.g., a medical records system of a health care provider that stores medical history information for populations of patients), one or more servers outside of system 10, and/or other sources of information. In some embodiments, external resources 16 include components that facilitate communication of information such as a network (e.g., the internet), electronic storage, equipment related to Wi-Fi technology, equipment related to Bluetooth® technology, data entry devices, sensors, scanners, and/or other resources. External resources 16 may be configured to communicate with processor 20, computing devices 18, electronic storage 50, and/or other components of system 10 via wired and/or wireless connections, via a network (e.g., a local area network and/or the internet), via cellular technology, via Wi-Fi technology, and/or via other resources. In some embodiments, some or all of the functionality attributed herein to external resources 16 may be provided by resources included in system 10.

Computing devices 18 are configured to provide interfaces between patients 12, caregivers 14 (e.g., doctors, nurses, friends, family members, administrators, staff members, technicians, etc.), and/or other users, and system 10. In some embodiments, individual computing devices 18 are and/or are included in desktop computers, laptop computers, tablet computers, smartphones, and/or other computing devices associated with individual caregivers 14, individual patients 12, and/or other users. In some embodiments, individual computing devices 18 are, and/or are included in equipment used in hospitals, doctor's offices, and/or other medical facilities to monitor patients 12; test equipment; equipment for treating patients 12; data entry equipment; and/or other devices. Computing devices 18 are configured to provide information to and/or receive information from caregivers 14, patients 12, and/or other users. For example, computing devices 18 are configured to present a graphical user interface 40 to caregivers 14 to facilitate entry and/or selection of prediction criteria (e.g., as described below). In some embodiments, graphical user interface 40 includes a plurality of separate interfaces associated with computing devices 18, processor 20, and/or other components of system 10; multiple views and/or fields configured to convey information to and/or receive information from caregivers 14, patients 12, and/or other users; and/or other interfaces.

In some embodiments, computing devices 18 are configured to provide graphical user interface 40, processing capabilities, databases, electronic storage, and/or other resources to system 10. As such, computing devices 18 may include processors 20, electronic storage 50, external resources 16, and/or other components of system 10. In some embodiments, computing devices 18 are connected to a network (e.g., the internet). In some embodiments, computing devices 18 do not include processors 20, electronic storage 50, external resources 16, and/or other components of system 10, but instead communicate with these components via the network. The connection to the network may be wireless or wired. For example, processor 20 may be located in a remote server and may wirelessly cause display of graphical user interface 40 to a caregiver 14 on a computing device 18 associated with caregiver 14 and/or to a patient 12 on a computing device 18 associated with patient 12. As described above, in some embodiments, an individual computing device 18 is a laptop, a personal computer, a smartphone, a tablet computer, and/or other computing devices. Examples of interface devices suitable for inclusion in an individual computing device 18 include a touch screen, a keypad, touch sensitive and/or physical buttons, switches, a keyboard, knobs, levers, a display, speakers, a microphone, an indicator light, an audible alarm, a printer, and/or other interface devices. The present disclosure also contemplates that an individual computing device 18 includes a removable storage interface. In this example, information may be loaded into a computing device 18 from removable storage (e.g., a smart card, a flash drive, a removable disk) that enables the caregivers 14, patients 12, and/or other users to customize the implementation of computing devices 18 and/or system 10. Other exemplary input devices and techniques adapted for use with computing devices 18 include, but are not limited to, an RS-232 port, RF link, an IR link, a modem (telephone, cable, etc.) and/or other devices.

Processor 20 is configured to provide information processing capabilities in system 10. As such, processor 20 may comprise one or more of a digital processor, an analog processor, a digital circuit designed to process information, an analog circuit designed to process information, a state machine, and/or other mechanisms for electronically processing information. Although processor 20 is shown in FIG. 1 as a single entity, this is for illustrative purposes only. In some embodiments, processor 20 may comprise a plurality of processing units. These processing units may be physically located within the same device (e.g., a server), or processor 20 may represent processing functionality of a plurality of devices operating in coordination (e.g., one or more servers, one or more computing devices 18 associated with patients 12 and/or caregivers 14, devices that are part of external resources 16, electronic storage 50, and/or other devices.)

In some embodiments, processor 20, external resources 16, computing devices 18, electronic storage 50, and/or other components may be operatively linked via one or more electronic communication links. For example, such electronic communication links may be established, at least in part, via a network such as the Internet, and/or other networks. It will be appreciated that this is not intended to be limiting, and that the scope of this disclosure includes embodiments in which these components may be operatively linked via some other communication media. In some embodiments, processor 20 is configured to communicate with external resources 16, computing devices 18, electronic storage 50, and/or other components according to a client/server architecture, a peer-to-peer architecture, and/or other architectures.

As shown in FIG. 1, processor 20 is configured via machine-readable instructions to execute one or more computer program components. The one or more computer program components may comprise one or more of a training information component 22, a criteria component 24, a model component 26, a prediction component 28, a display component 30, and/or other components. Processor 20 may be configured to execute components 22, 24, 26, 28, and/or 30 by software; hardware; firmware; some combination of software, hardware, and/or firmware; and/or other mechanisms for configuring processing capabilities on processor 20.

It should be appreciated that although components 22, 24, 26, 28, and 30 are illustrated in FIG. 1 as being co-located within a single processing unit, in embodiments in which processor 20 comprises multiple processing units, one or more of components 22, 24, 26, 28, and/or 30 may be located remotely from the other components. The description of the functionality provided by the different components 22, 24, 26, 28, and/or 30 described below is for illustrative purposes, and is not intended to be limiting, as any of components 22, 24, 26, 28 and/or 30 may provide more or less functionality than is described. For example, one or more of components 22, 24, 26, 28, and/or 30 may be eliminated, and some or all of its functionality may be provided by other components 22, 24, 26, 28, and/or 30. As another example, processor 20 may be configured to execute one or more additional components that may perform some or all of the functionality attributed below to one of components 22, 24, 26, 28, and/or 30.

Training information component 22 is configured to obtain training information. The training information is related to patients 12. The training information related to patients indicates health information for a plurality of patients 12 and/or other information. In some embodiments, the training information comprises demographic information indicating demographics associated with patients 12, vital signs information indicating vital signs associated with patients 12, medical condition information indicating medical conditions experienced by patients 12, treatment information indicating treatments received by patients 12, outcome information indicating health outcomes for patients 12, and/or other training information. In some embodiments, the training information includes a plurality of (e.g., original—as described above) features associated with the individual types of information described above and/or other types of information. For example, the training information may include demographic features (e.g., gender, ethnicity, age, etc.) associated with demographics of patients 12, vital signs features (e.g., heart rate, temperature, respiration rate, etc.) associated with vital signs associated with patients 12, medical condition features (e.g., a disease type, symptoms, behaviors, etc.) associated with medical conditions experienced by patients 12, treatment features (e.g., length of treatment, length of stay in a medical facility, medications, interventions, etc.) associated with treatments received by patients 12, outcome features (e.g., discharge date, prognosis, readmission date, etc.) associated with health outcomes for patients 12, and/or other training information. It should be noted that the example features described above are not intended to be limiting. As described above, an uncountable number of possible features exist and those listed above are a small subset of examples.

In some embodiments, the obtaining includes electronically importing the training information (e.g., from one or more databases included in external resources 16), facilitating entry and/or selection of the training information (e.g., via computing devices 18), uploading and/or downloading training information, receiving emails, texts, and/or other communications that include training information, and/or other activities. For example, in some embodiments, the training information is stored in one or more databases (e.g., such as electronic databases included in external resources 16), and obtained by training information component 22 from a database. For example, training information component 22 may obtain training information from medical records for a plurality of patients 12 which include information such as initial vital signs of patients 12, treatments provided to patients 12 with the respective initial vital signs, respective vital signs resulting from the treatments, overall health outcomes for the patients 12, and/or other information. In some embodiments, obtaining includes electronically importing only a portion and/or a subset of the training information (e.g., only information associated with specific features, etc.) from one or more databases. In some embodiments, the portion and/or subset may be determined at manufacture of system 10, determined by a user (e.g., a caregiver 14 and/or a patient 12) via a user interface 40 of a computing system 18, and/or by other methods.

In some embodiments, training information component 22 is configured to obtain additional training information. In some embodiments, the additional training information is obtained continuously on a periodic basis (e.g., at predetermined intervals), in accordance with a schedule, or based on other automated triggers (e.g., responsive to identification of new patients 12). In some embodiments, the frequency with which training component 22 obtains the additional training information is set at manufacture, set and/or adjusted by users via a user interface 40 of a computing device 18, and/or by other methods. The additional training information comprises additional demographics information, additional vital signs information, additional medical conditions information, additional treatment information, additional outcome information, and/or other information. For example, one or more of the databases included in external resources 16 may be updated with additional information as additional patients are treated at medical facilities, the same patients continue to be treated and/or are retreated, test results are added, patient outcomes are determined, etc. In some embodiments, training information component 22 may be configured to update such databases (e.g., continuously on a periodic basis, in accordance with a schedule, or based on other automated triggers). In some embodiments, training information component 22 may be configured to update such databases with information generated by system 10 (e.g., as described below). For example, training information component 22 may be configured to update such databases with information indicating that system 10 predicted a high likelihood of death for a given patient 12. In some embodiments, the prediction information may be used by training information component 22 in combination with updated information (e.g., indicating whether the given patient died) to determine whether the prediction made by system 10 was accurate. In some embodiments, this prediction accuracy information is included in the additional training information.

Criteria component 24 is configured to obtain prediction criteria. The prediction criteria convey the expectations of a user (e.g., a caregiver 14) for information used by a prediction model (described below) to generate a prediction related to a health outcome for a given patient 12. In some embodiments, the prediction related to a health outcome for the given patient 12 is risk of readmission to a medical facility, mortality risk, length of stay, hospital-acquired infection risk, and/or other health outcome predictions. The prediction criteria are obtained via user (e.g., caregiver 14) input entered and/or selected via a user interface 40 of a computing device 18 associated with the user and/or other devices. In some embodiments, criteria component 24 facilitates entry and/or selection of the prediction criteria via one or more views of user interface 40 that include one or more fields for viewing, entering, and/or selecting information (e.g., criteria). The prediction criteria are used to generate a prediction model (described below) and the patient-related health outcome predictions.

In some embodiments, the prediction criteria include constraints on which features may be used in the prediction model, constraints on how may features may be used in the prediction model, and/or other information. In some embodiments, the prediction criteria include which prediction-contributing features are to be used by the prediction model for generating patient-related health outcome predictions. In some embodiments, the prediction criteria include a target false positive outcome prediction rate associated with a given feature, a target degree of correlation between the given feature and the prediction related to the health outcome, a target amount the given feature influences the prediction related to the health outcome relative to other features, a type of feature, and/or other criteria. For example, criteria component 24 is configured such that, using system 10, a user can specify constraints (e.g., criteria) such as (i) the highest false positive rate (e.g., the number of patients falsely classified as positive) should be less than a predetermined threshold value (e.g., 20%) for a given feature, (ii) a positive association should be maintained between a given feature and the prediction, (iii) a given feature should be used only if it yields a threshold (e.g., 2%) gain in prediction accuracy over the use of another feature and/or features, (iv) the model should only use features of a specific type, such as actionable features (e.g., features wherein the actions of a caregiver 14 may change the feature value such as choice of treatment), versus non-actionable features (e.g., age, gender, etc.), (v) the model should only use two (this example is not intended to be limiting and may be any number that allows system 10 to function as described herein) features, and/or other constraints.

Model component 26 is configured to generate a prediction model. The prediction model is generated based on the prediction criteria, the training information, and/or other information. The training information and the prediction criteria are used by model component 26 to generate the prediction model subject to the prediction criteria. In some embodiments, the prediction model is generated by minimizing a 0-1 loss function for accuracy and a L0 norm regulation for sparsity subject to the prediction criteria using a constraint programming optimization algorithm. In some embodiments, model component 26 is configured to train the prediction model using the training information and/or other information.

In some embodiments, the prediction model may be and/or include a neutral network that is trained and utilized for generating predictions (described below). As an example, neural networks may be based on a large collection of neural units (or artificial neurons). Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neutral unit itself) may have a threshold function such that the signal must surpass the threshold before it is allowed to propagate to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free-flowing, with connections interacting in a more chaotic and complex fashion.

By way of a non-limiting example, in some embodiments, model component 26 is configured to receive prediction criteria from criteria component 24 for a prediction model that is to be generated and used to generate a health outcome prediction for a given patient 12. In this example, the health outcome prediction may be predicted risk of readmission, and/or other health outcome predictions. Continuing with this example, the prediction criteria from a caregiver 14 may specify that only two features are to be used. While the caregiver 14 may specify two specific features to be used for the model such as “age” and “length of stay,” the caregiver may just specify that two features are to be used, and the model determines which two features provide the best prediction based upon the training data and any constraints specified by the caregiver. Model component 26 is configured to process the prediction criteria and the training information (described above) such that the generated prediction model for risk of readmission satisfies the prediction criteria. In this example, using the 0-1 loss function for accuracy and the L0 norm regularization for sparsity (subject to any constraints on accuracy and/or sparsity in the prediction criteria) are minimized by model component 26 using a constraint programming optimization algorithm to generate a predictive model for risk of readmission for a given patient 12. For example, the following risk score model for readmission using the above mentioned criteria may be generated (which indicates for example, that “age” (multiplier of 3) is more influential than “length of stay” (multiplier of 2) on risk of readmission).

Risk of readmission=3*Age (years) +2*Length of Stay (days) −102 (1)

In some embodiments, the Age and Length of Stay features may be determined by the training process or specifically chosen by the caregiver 14. In some embodiments (e.g., when training information component 22 obtains additional training information indicating whether prior predictions were accurate, etc.), model component 26 updates the prediction model based on the prediction criteria, the additional training information, and/or other information (e.g., continuously on a periodic basis, in accordance with a schedule, or based on other automated triggers). In some embodiments, the updating is performed responsive to an indication made by a user (e.g., caregiver 14) via a user interface 40, at regular time intervals (e.g., programmed at manufacture, set and/or adjusted via a user interface 40), and/or at other times.

In some embodiments, model component 26 is configured to use other optimization techniques (e.g., for risk management), to generate predictive models and/or health outcome predictions (e.g., readmission risk predictions) for populations of patients 12 at risk for readmission and/or other health outcomes (e.g., instead of and/or in addition to re-admission) after experiencing other medical conditions (e.g., acquiring a healthcare-associated infection, such as methicillin-resistant Staphylococcus aureus or other multi-drug resistant species). For example, system 10 may be configured to facilitate identifying patients 12 that should be observed with greater oversight, flagging patients for follow-up or further testing using high-resolution genomic sequencing-based assays, such that patients 12 with persistent infections (for example) may be identified more rapidly, providing timely and appropriate treatment to shorten the continuum of care, saving lives and saving costs to healthcare providers, for example.

In some embodiments, model component 26 may be configured to apply interpretable machine learning algorithms to identify salient features that may be used for clinical decision support, through normalizing discrete and/or categorical independent variables within the predictive model for relevant feature selection. For example, when using both clinical information (such as location of stay, device, caretaker, etc.) and genomics-based variables, difficult to resolve prediction of antibiotic resistance or sensitivity may be achieved.

Prediction component 28 is configured to generate a prediction. The prediction is generated based on the prediction model, patient information associated with a given patient 12 for whom the prediction is generated, and/or other information. The patient information associated with the given patient 12 indicates health information for the given patient 12 and/or other information. For example, the patient information for given patient 12 may comprise demographic information for given patient 12, levels of vital signs for given 12, medical condition information indicating medical conditions experienced by given patient 12, treatment information indicating treatments received by given patient 12, and/or other patient information. The prediction is a prediction related to a health outcome of the given patient 12. For example, continuing with the example above prediction component 28 is configured to input the age (e.g., a feature used in the predictive model based on the prediction criteria as described above) of given patient 12 and the number of days (e.g., a second feature used in the predictive model) given patient 12 was in a medical facility into Equation 1 (e.g., the predictive model) above to determine a score indicative of the risk of readmission (e.g., a prediction related to a health outcome) of given patient 12 back into the medical facility.

Display component 30 is configured to cause display of the prediction related to the health outcome for the given patient 12. In some embodiments, the prediction is displayed via user interface 40 of a computing device 18 associated with a patient 12, a caregiver 14, and/or other users. In some embodiments, the display comprises graphical, textual, or other representations; provision of one or more textual and/or graphical fields in various views of graphical user interface 40; and/or other presentation. In some embodiments, the display of the prediction for the given patient includes a numerical indication of the health outcome prediction (e.g., a risk of readmission score), a list of the two or more prediction-contributing features (e.g., the features used in the prediction model such as “age” and “length of stay” in the example above), a mathematical relationship between the two or more prediction-contributing features (e.g., Equation 1 above), values of accuracy metrics corresponding to the two or more prediction-contributing features (e.g., sensitivity, specificity, area under the receiver operating characteristic curve (AUC), an F1 score, etc.), a chart, graph, table, and/or plot, and/or other information.

In some embodiments, display component 30 is configured to display predictions related to health outcomes of a plurality of patients 12 (e.g., responsive to the components of processor 20 generating predictions for the other patients 12 as described above). In some embodiments, the display of the prediction for the given patient 12 and/or the predictions for the plurality of patients 12 (e.g., via one or more user interfaces 40 on one or more computing devices 18 associated with caregivers 14 and/or patients 12) comprises a scaled display of two or more of the prediction-contributing features used by the prediction model for generating the patient-related predictions relative to each other such that any change in a value of one of the two or more prediction-contributing features causes a corresponding scaled change in values of the others of the two or more prediction-contributing features.

For example, the scaled display may comprise a scatter plot, a chart, a histogram, a table, and/or other displays. Display component 30 may be configured such that caregiver 14 and/or other users may scale (e.g., using graphical user interface 40 and/or other components of a computing device 18 associated with caregiver 14) the two or three (for example) selected features (features used in the prediction model) for better visualization, where a one-unit increase (for example) of the value of an individual scaled feature would change the values of other selected features, and the health outcome prediction (e.g., risk of re-admission) by the same amount. Continuing with the example above, the features “age” and “length of stay” may be scaled such that a one-unit increase in either one of these scaled features would cause an increase in the other one of these features, and the risk of readmission, by the same amount. In this example, display component 30 may be configured to display age/3 (e.g., a scaled feature) and/or length of stay/2 (e.g., another scaled feature) such that a one-unit increase (for example) of the value of an individual scaled feature (e.g., age/3 and/or length of stay/2) would change the values of the other feature, and the risk of re-admission by the same amount.

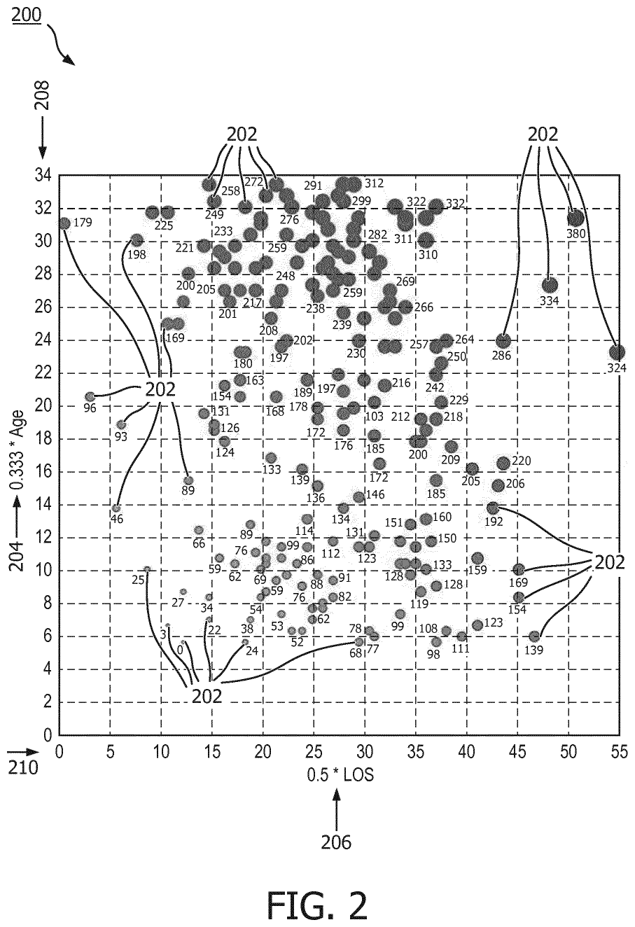

By way of a non-limiting example, FIG. 2 illustrates a scatter plot 200 of readmission risk values 202 (health outcome predictions) for a plurality of patients 12 (FIG. 1). As shown in FIG. 2, the features “age” 204 and “length of stay” (LOS) 206 are shown on Y-axis 208 and X-axis 210 respectively. Age 204 and LOS 206 are scaled such that a one-unit increase in either one of these scaled features 204, 206 causes an increase in the other one of these features, and the risk of readmission 202, by the same amount. In this example, display component 30 (FIG. 1) has displayed 0.33 * Age (or age/3) and 0.5* LOS (or LOS/2) based on Equation 1 used in the example above such that a one-unit increase (for example) of the value of an individual scaled feature (e.g., 0.33 * age and/or 0.5 * LOS) would change the risk of re-admission by the same amount.

Returning to FIG. 1, in some embodiments, the prediction (e.g., from prediction component 28) related to the health outcome of the given patient 12 further comprises an initial prediction related to the health outcome presented via, for example, user interface 40 before presentation of the scaled display and/or the other information described above. In such embodiments, the initial prediction related to the health outcome of the given patient 12 comprises the numerical indicator of the health outcome prediction (e.g., the risk of readmission score), the list of the two or more prediction-contributing features, the mathematical relationship between the two or more prediction-contributing features, the values of the accuracy metrics corresponding to the two or more prediction-contributing features, and/or other information.

In such embodiments, display component 30 is configured to cause presentation of the initial prediction related to the health outcome of the given patient 12 via user interface 40 (for example), facilitate review of the initial prediction by a caregiver 14 and/or other users, facilitate receipt of refined prediction criteria, and update the prediction model based on the refined prediction criteria, the training information (or additional training information), and/or other information. The refined prediction criteria comprise indications of whether to include and/or exclude new and/or different individual features relative to features indicated by the original prediction criteria, an adjusted target false positive outcome prediction rate associated with a given feature, an adjusted target degree of correlation between the given feature and the prediction related to the health outcome, an adjusted target amount the given feature influences the prediction related to the health outcome relative to other features, and/or other adjusted criteria. For example, a physician (e.g., a caregiver 14) may decide that for a given patient 12, after reviewing previously entered criteria, the prediction model should not use “age” and “length of stay,” and these criteria may be excluded from consideration by the model via constraints. Alternatively, the features “gender” and “admission diagnosis,” and/or that three features should be used instead of two may be specified by the caregiver 14. There are many more examples of changes to the criteria that could be made by the caregiver 14.

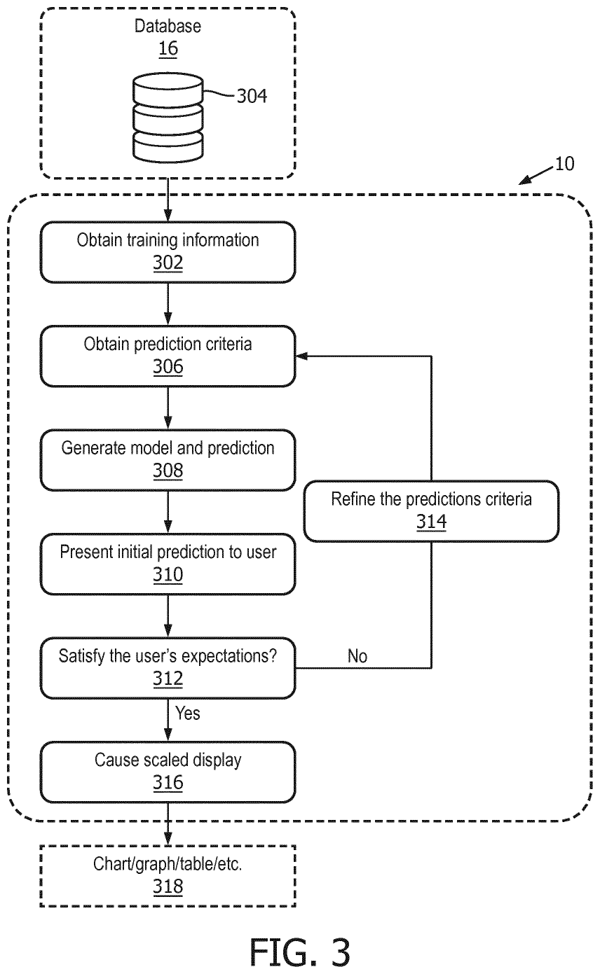

By way of a non-limiting example, FIG. 3 illustrates operations performed by system 10. As shown in FIG. 3, at an operation 302, training information is obtained from database 304 of external resources 16. At an operation 306, prediction criteria are obtained. The prediction criteria may be obtained from a user (e.g., caregiver 14 shown in FIG. 1) via a user interface 40 (FIG. 1) of a computing device 18 (FIG. 1) associated with the user, for example. In the example described above, the caregiver 14 may decide to select risk of readmission as the prediction criteria. Also, the caregiver 14 determines the number of features to be considered in the model. In the example above this is two, but higher numbers of features may also be selected. Further, the caregiver 14 may provide additional constraints. For example, the caregiver may understand that certain features have more influence on risk of readmission than others, e.g., age has a greater effect then length of stay, hence the model will be constrained such that age has greater effect on the risk of readmission than length of stay. Other constraints may be defined as well, such as (i) the highest false positive rate (e.g., the number of patients falsely classified as positive) should be less than a predetermined threshold value (e.g., 20%) for a given feature, (ii) a positive association should be maintained between a given feature and the prediction, (iii) a given feature should be used only if it yields a threshold (e.g., 2%) gain in prediction accuracy over the use of another feature and/or features, (iv) the model should only use features of a specific type, such as actionable features (e.g., features wherein the actions of a caregiver 14 may change the feature value such as choice of treatment), versus non-actionable features (e.g., age, gender, etc.), etc. Other prediction criteria may be specified as well, with the caregiver 14 providing constraints on the model based upon their knowledge and experience.

At an operation 308, the prediction model and a prediction may be generated. This may include generating the prediction model based on the prediction criteria, the training information, and/or other information (as described above); generating the prediction based on the prediction model and patient information associated with a given patient; and/or other operations. A machine learning model is used to determine the specific features to use in the prediction model. This may be done using L0 norm regularization, which leads to a model of the form pred val=Σᵢ₌₀^{N-1} αᵢ·xᵢ+b, where pred val is the desired predicted value, xᵢ is a predictive-value feature, αᵢ is a predictive-value feature scale factor, and b. The machine learning model uses the training data and the user specified constraints, including the number of predictive-value features or even specific features to use, to determine which predictive-value features xᵢ provide the best predictive value. The machine also determines the predictive-value feature scale factors αᵢ for each predictive-value feature and the bias b. This results in an optimized linear prediction model based upon the best predictive-value features. The optimization may use a 0-1 loss function to determine the accuracy of the L0 norm regularization to determine the various values for the predictive model. The predictive-value feature scale factors αᵢ (i.e., the inverse there of) may be used to scale the display outputs as shown in FIG. 2.

At an operation 310, an initial prediction model is presented to a user via, for example, user interface 40. In some embodiments, the initial prediction is related to the health outcome of a given patient 12 (FIG. 1) and comprises a numerical indicator of the health outcome prediction (e.g., the risk of readmission score), a list of the prediction-contributing features, a mathematical relationship between the prediction-contributing features, values of accuracy metrics corresponding to the prediction-contributing features, and/or other information.

At an operation 312, system 10 causes presentation of the initial prediction related to the health outcome of the given patient (e.g., patient 12), and facilitates review (e.g., via user interface 40 shown in FIG. 1) of the initial prediction by a user (e.g., caregiver 14). At an operation 314, system 10 facilitates receipt of refined prediction criteria, and updates the prediction model based on the refined prediction criteria, the training information (or additional training information), and/or other information (e.g. operations 306-310 may be repeated one or more times).

At an operation 316, system 10 is configured to cause a display 318 of the prediction related to the health outcome for the given patient (patient 12). In some embodiments, the prediction is displayed via user interface 40 of a computing device 18 associated with a patient 12, a caregiver 14, and/or other users. In some embodiments, system 10 is configured to display predictions related to health outcomes of a plurality of patients. In some embodiments, display 318 comprises a scaled display of two or more of the prediction-contributing features used by the prediction model for generating the patient-related predictions relative to each other such that on the display a unit change in a value of one of the two or more prediction-contributing features causes the same change in the predicted value.

Returning to FIG. 1, electronic storage 50 comprises electronic storage media that electronically stores information (e.g., criteria, mathematical equations, predictions, etc.). The electronic storage media of electronic storage 50 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 50 may be (in whole or in part) a separate component within system 10, or electronic storage 50 may be provided (in whole or in part) integrally with one or more other components of system 10 (e.g., computing devices 18, processor 20, etc.). In some embodiments, electronic storage 50 may be located in a server together with processor 20, in a server that is part of external resources 16, in a computing device 18, and/or in other locations. Electronic storage 50 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 50 may store software algorithms, information determined by processor 20, information received via a computing device 18 and/or graphical user interface 40 and/or other external computing systems, information received from external resources 16, and/or other information that enables system 10 to function as described herein.

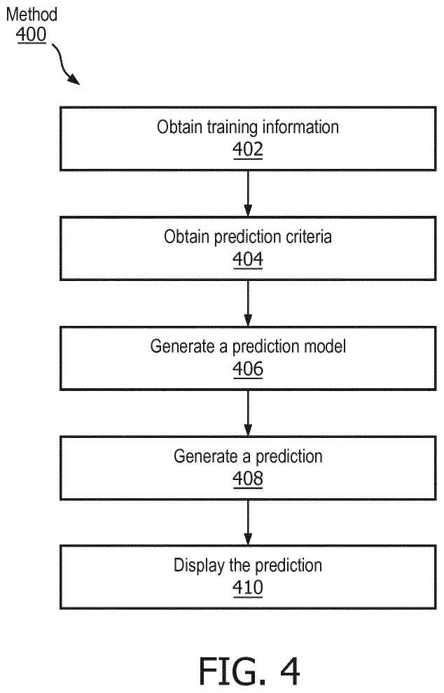

FIG. 4 illustrates a method 400 for providing user-customized prediction models and health-related predictions based thereon with a prediction system. The system comprises one or more hardware processors and/or other components. The one or more hardware processors are configured by machine readable instructions to execute computer program components. The computer program components include a training information component, a criteria component, a model component, a prediction component, a display component, and/or other components. The operations of method 400 presented below are intended to be illustrative. In some embodiments, method 400 may be accomplished with one or more additional operations not described, and/or without one or more of the operations discussed. Additionally, the order in which the operations of method 400 are illustrated in FIG. 4 and described below is not intended to be limiting.

In some embodiments, method 400 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 400 in response to instructions stored electronically on an electronic storage medium. The one or more processing devices may include one or more devices configured through hardware, firmware, and/or software to be specifically designed for execution of one or more of the operations of method 400.

At an operation 402, training information is obtained. The training information is related to patients. In some embodiments, the training information comprises demographic information indicating demographics associated with the patients, vital signs information indicating vital signs associated with the patients, medical condition information indicating medical conditions experienced by the patients, treatment information indicating treatments received by the patients, outcome information indicating health outcomes for the patients, and/or other training information. In some embodiments, operation 402 is performed by a processor component the same as or similar to training information component 22 (shown in FIG. 1 and described herein).

At an operation 404, prediction criteria are obtained. The prediction criteria are obtained via user input entered and/or selected via a user interface and/or other devices. The prediction criteria are used by a prediction model for generating patient-related predictions. The prediction criteria include which and how many prediction-contributing features are to be used by the prediction model for generating patient-related predictions. In some embodiments, the prediction criteria include a target false positive outcome prediction rate associated with a given feature, a target degree of correlation between the given feature and the prediction related to the health outcome, a target amount the given feature influences the prediction related to the health outcome relative to other features, and/or other criteria. In some embodiments, operation 404 is performed by a processor component the same as or similar to criteria component 24 (shown in FIG. 1 and described herein).

At an operation 406, a prediction model is generated. The prediction model is generated based on the prediction criteria, the training information, and/or other information. In some embodiments, the prediction model is generated by minimizing a 0-1 loss function for accuracy and a L0 norm regulation for sparsity subject to the prediction criteria. In some embodiments, operation 402 includes obtaining additional training information (e.g., continuously on a periodic basis, in accordance with a schedule, or based on other automated triggers). The additional training information comprises additional demographics information, additional vital signs information, additional medical conditions information, additional treatment information, or additional outcome information. In such embodiments, the prediction model is updated based on the prediction criteria and the additional training information. In some embodiments, operation 406 is performed by a processor component the same as or similar to model component 26 (shown in FIG. 1 and described herein).

At an operation 408, a prediction is generated. The prediction is generated based on the prediction model and patient information associated with a patient. The prediction is a prediction related to a health outcome of the patient. In some embodiments, operation 408 is caused by a processor component the same as or similar to prediction component 28 (shown in FIG. 1 and described herein).

At an operation 410, the prediction is displayed. In some embodiments, operation 410 includes generating, based on the prediction model and patient information associated with other patients, predictions related to health outcomes of the other patients; and causing display of the prediction and the other predictions on the user interface. In some embodiments, the display of the prediction and the other predictions on the user interface comprises a scaled display of two or more of the prediction-contributing features used by the prediction model for generating the patient-related predictions relative to each other such that any change in a value of one of the two or more prediction-contributing features causes a corresponding scaled change in values of the others of the two or more prediction-contributing features.

In some embodiments, the prediction (e.g., from operation 408) related to the health outcome further comprises an initial prediction related to the health outcome presented via the user interface before the scaled display. In such embodiments, the initial prediction related to the health outcome comprises a numerical outcome risk score, a list of the two or more prediction-contributing features, a mathematical relationship between the two or more prediction-contributing features, values of accuracy metrics corresponding to the two or more prediction-contributing features, and/or other information. In such embodiments, operation 408 further comprises causing presentation of the initial prediction related to the health outcome via the user interface, facilitating receipt of refined prediction criteria, and updating the prediction model based on the refined prediction criteria, the training information (or additional training information), and/or other information. In some embodiments, operation 410 is caused by a processor component the same as or similar to display component 30 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A system configured to provide user-customized prediction models and health-related predictions based thereon, the system comprising one or more hardware processors configured by machine readable instructions to:
obtain training information related to patients, the training information comprising one or more of demographic information indicating demographics associated with the patients, vital signs information indicating vital signs associated with the patients, medical condition information indicating medical conditions experienced by the patients, treatment information indicating treatments received by the patients, or outcome information indicating health outcomes for the patients;
obtain, via a user interface, a user input indicating prediction criteria that are to be used by a prediction model for generating patient-related predictions, the prediction criteria including which and how many prediction-contributing features are to be used by the prediction model for generating patient-related predictions;
generate the prediction model based on (i) the prediction criteria and (ii) the training information, and
generate based on the prediction model and patient information associated with a patient, a prediction related to a health outcome of the patient.

2. (canceled)

3. The system of claim 8, wherein the one or more hardware processors are further configured to: cause presentation of the initial prediction related to the health outcome via the user interface, facilitate receipt of refined prediction criteria, and update the prediction model based on (i) the refined prediction criteria and (ii) the training information.

4. The system of claim 1, wherein the one or more hardware processors are further configured:
obtain additional training information; the additional training information comprising additional demographics information, additional vital signs information, additional medical conditions information, additional treatment information., or additional outcome information; and
update the prediction model based on (i) the prediction criteria and (ii) the additional training information.

5. (canceled)

6. The system of claim 1, wherein the one or more hardware processors are configured such that the prediction model is generated by minimizing a 0-1 loss function for accuracy and a L0 norm regulation for sparsity subject to the prediction criteria.

7. The system of claim 1, wherein the one or more hardware processors are further configured:
generate, based on the prediction model and patient information associated with other patients, predictions related to health outcomes of the other patients; and
cause display of the prediction and the other predictions on the user interface,
wherein the display of the prediction and the other predictions on the user interface comprises a scaled display of two or more of the prediction-contributing features used by the prediction model for generating the patient-related predictions relative to each other such that any change in a value of one of the two or more prediction-contributing features causes a corresponding scaled change in values of the others of the two or more prediction-contributing features.

8. The system of claim 7, wherein the one or more hardware processors are further configured such that the prediction related to the health outcome comprises an initial prediction related to the health outcome presented via the user interface before the scaled display, the initial prediction related to the health outcome comprising a numerical outcome risk score, a list of the prediction-contributing features, a mathematical relationship between the prediction-contributing features, and values of accuracy metrics corresponding to the two or more prediction-contributing features.

9. The of claim 1, wherein the one or mote hardware processors are configured such that the prediction criteria further include a target false positive outcome prediction rate associated with a given feature, a target degree of correlation between the given feature and the prediction related to the health outcome, or a target amount the given feature influences the prediction related to the health outcome relative to other features.

10. A method for providing user-customized prediction models and health-related predictions based thereon with a prediction system, the system comprising one or more hardware processors configured by machine readable instructions, the method comprising
Obtaining, with the one or more hardware processors, training information related to patients, the training information; comprising one or more of demographic information indicating demographics associated with the patients, vital signs information indicating vital signs associated with the patients, medical condition information indicating medical conditions experienced by the patients, treatment information indicating treatments received fay the patients, or outcome information indicating health outcomes for the patients;
obtaining, with the one or more hardware processors via a user interface, a user input indicating prediction criteria that are to be used by a prediction model for generating patient-related predictions, the prediction criteria including which and how many prediction-contributing features are to be used by the prediction model for generating patient-related predictions;
generating, with the one or more hardware processors, the prediction model based on (i) the prediction criteria and (ii) the training information, and
generating, with the one or more hardware processors, based on the prediction model and patient information associated with a patient, a prediction related to a health outcome of the patient.

11. (canceled)

12. The method of claim 1, further comprising:
causing, with the one or more hardware processors, presentation of the initial prediction related to the health outcome via the user interface,
facilitating, with the one or more hardware processors receipt of refined prediction criteria, and
updating, with the one or more hardware processors, the prediction model based on (i) the refined prediction criteria and (ii) the training information.

13. The method of claim 10, further comprising:
obtaining, with the one or more hardware processors, additional training information, the additional training information comprising additional demographics information, additional vital signs information, additional medical conditions information, additional treatment information, or additional outcome information; and
updating, with the one or more hardware processors, the prediction model based on (i) the prediction criteria and (ii) the additional updated updated training information.

14. (canceled)

15. The method of claim 10, wherein the prediction model is generated by minimizing a 0-1 loss function for accuracy and a L0 norm regulation for sparsity subject to the prediction criteria.

16. The method of claim 10, further comprising:
generating, with the one or more hardware processors, based on the prediction model and patient information associated with other patients, predictions related to health outcomes of the other patients; and
cause display of the prediction and the other predictions on the user interface,
wherein the display of the prediction and the other predictions on the user interface comprises on a scaled display of two or more of the prediction-contributing features used by the prediction model for generating the patient-related predictions relative to each other such that any change in a value of one of the two or more prediction-contributing features causes a corresponding scaled change in values of the others of the two or more.

17. The method of claim 16, wherein the prediction related to the health outcome comprises an initial prediction related to the health outcome presented via the user interface before the scaled display, the initial prediction related to the health outcome comprising a numerical outcome risk score, a list of the prediction-contributing features, a mathematical relationship between the prediction-contributing features, and values of accuracy metrics corresponding to the two or more prediction-contributing features.

18. The method of claim 10, wherein the prediction criteria further include one of a target false positive outcome prediction rate associated with a given feature, a target degree of correlation between the given feature and the prediction related to the health outcome, or a target amount the given feature influences the prediction related to the health outcome relative to other features.
